(19) United States
(12) Patent Application Publication
(10) Pub. No.: US 2009/0221981 A1
(43) Pub. Date: Sep. 3, 2009

(54) RELEASABLE NONWOVEN WRAPPER FOR AN ABSORBENT ARTICLE

(76) Inventor: Richmond R. Cohen, Williamsport, PA (US)

Correspondence Address: AMSTER, ROTHSTEIN & EBENSTEIN LLP, 90 PARK AVENUE, NEW YORK, NY, 10016, US

(21) Appl. No.: 12/093,022

(22) PCT Filed: Nov. 8, 2006
(86) PCT No.: PCT/US06/43418

§ 371 Date: Jan. 13, 2009

Related U.S. Application Data
(60) Provisional application No. 60/734,926, filed on Nov. 9, 2005.

(51) Int. Cl. A61F 13/15; A61B 19/00

(52) U.S. Cl. 604/385.02; 604/385.23; 206/440

(57) ABSTRACT

An absorbent article and wrapper, where the absorbent article has a topsheet, absorbent core and backsheet, and an adhesive layer is applied to the backsheet. The wrapper has an internal and external layer, where the internal layer is made from a nonwoven having low fluid permeability and is treated with a release agent.

### TECHNICAL FIELD

The present invention relates to wrappers for absorbent articles and, more particularly, to wrappers made from nonwoven materials incorporating release coatings.

### BACKGROUND

For convenience, discreetness and protection, some disposable absorbent articles such as bladder control pads, pantiliners and sanitary napkins are packaged individually within a larger sized bag or box. The individual packages or wrappers for these products provide a convenient means for a user (such as a woman) to carry an individual product or two in her pocketbook or purse. The wrapper provides protection for the hygienic pad from contaminants that could otherwise contact the product. Typically these hygienic pads have an adhesive on one side so that the pad may be secured to the user's underwear.

Most of the wrappers seen on the market today consist of a polyethylene film wrapped around an absorbent pad that is bi-folded or tri-folded. The film is sealed along two of its edges and generally is sealed by adhesive in one spot that occurs centrally across the open seam between the sealed edges. Upon opening the seal along the seam, the pad can be removed from the wrapper. Then, a siliconized release paper is removed from the adhesive strip on the pad, exposing the adhesive which enables the user to secure the absorbent pad to her underwear to keep the pad in place.

Although such film wrappers do provide protection for the absorbent pad, the film wrapper will crinkle when handled, and this noise diminishes the discreetness of the product. Furthermore, to deploy the absorbent pad, the user needs to remove and dispose of both the wrapper material plus the siliconized release paper. These efforts are less convenient than removing and disposing of only one material rather than two.

U.S. Pat. No. 6,716,203 describes a laminated nonwoven/film overwrap. Such wrappers are quieter and softer, but the use of the product still requires removal of two materials, namely, the pouch and the siliconized release paper.

Siliconized film wrappers are described in U.S. Pat. No. 4,556,146. Such wrappers have the convenience of only removing and disposing of one material prior to using the absorbent pad. As indicated earlier, such wrappers do not possess the softness, quietness and overall discreetness of nonwoven wrappers.

Thus, what is needed is a discreet, quiet, soft and convenient wrapper material for individual wrapping of absorbent articles. Such a novel and useful material should also be convenient and practical to manufacture.

### SUMMARY OF THE INVENTION

An exemplary object of the present invention is a nonwoven wrapper for an absorbent article that combines the functions of both a wrapper and release paper. The nonwoven wrapper uses a low air permeability nonwoven material to which a release coating is applied.

In a preferred embodiment, a spunbond/meltblown laminate is used with the spunbond forming the exterior of the wrapper to provide softness and strength and the meltblown forming the interior layer for application of the release agent.

The present invention also encompasses the combination of a releasable nonwoven wrapper described above and an absorbent article.

### BRIEF DESCRIPTION OF THE DRAWINGS

Various exemplary embodiments of this invention will be described in detail, with reference to the following figures, wherein:

FIG. 1A is a sectional view of one embodiment of the invention having a single nonwoven layer.

FIG. 1B is a sectional view of one embodiment of the invention having two nonwoven layers.

FIG. 2 is a sectional view of one embodiment of the invention having an absorbent pad and wrapper.

FIG. 3. is a perspective view of one embodiment of the invention having an absorbent pad and wrapper.

### DETAILED DESCRIPTION

The present invention is a nonwoven wrapper having a releasable coating for use with an absorbent article having an adhesive layer. Specifically, the nonwoven wrapper is designed for use with absorbent pads, such as sanitary napkins and bladder control pads, although not limited solely to this use. For example, the nonwoven wrapper of the present invention could also be adapted to absorbent articles such as male guards, protective underwear, diapers and wipes. In the preferred embodiment using an absorbent pad, the nonwoven wrapper both wraps around the absorbent pad and adheres releasably to the adhesive strip on the pad. This wrapper material maximizes protection, convenience and discreetness.

The nonwoven of the present invention can be either a composite or a single component with a limited air permeability. A composite nonwoven contains two or more nonwoven layers. The nonwoven layers of the composite may be laminated to each other, that is, they may be secured together in some manner including adhesive bonding, thermal bonding, mechanical entanglement, ultrasonic bonding or other attachment means. Preferably, the nonwoven is a meltblown material or a composite of a meltblown material and a spunbond material. A particularly useful embodiment of the invention is a laminate of a spunbond material with a meltblown material. The spunbond material is resistant to abrasion, enhancing portability of the article, while the low permeability of the meltblown holds the adhesive and release coating applications in place, in addition to providing opacity. Other examples of suitable nonwovens include spunbond or carded nonwovens with fine denier fibers, wetlaid nonwovens or other nonwovens having low air permeability.

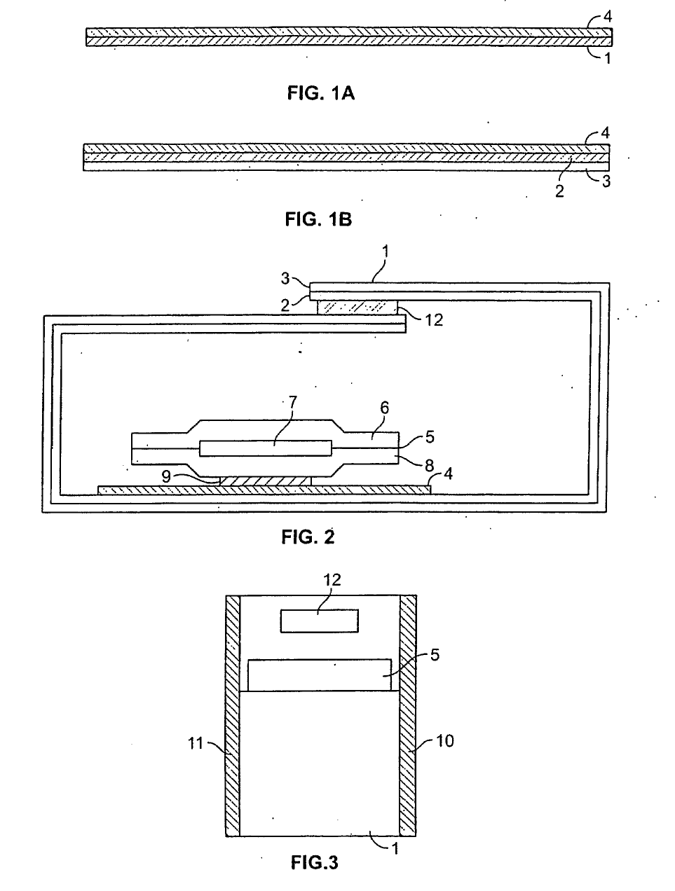

Two embodiments of the present invention are shown in FIGS. 1A and 1B. As shown in FIG. 1A, the wrapper material 1 is a single layer of nonwoven having a release coating 4 on one side. In a preferred embodiment, shown in FIG. 1B, the wrapper material 1 includes an inner layer 2, preferably made from meltblown fibers, coated with a release coating 4, preferably containing at least in part silicone chemistry. An outer layer 3, preferably made from spunbond fibers, is not treated.

As shown in FIG. 2, the portion of the wrapper containing the release coating 4 is releasably adhered to the adhesive strip 9 of an absorbent pad while the product is in storage. Typically, the absorbent pad 5 has a topsheet 6 covering an absorbent core 7 and backsheet 8. At least a portion of the backsheet 8 is coated with adhesive 9. When the absorbent pad 5 is folded into the wrapper 1, the portions of backsheet 8 coated with adhesive 9 come into contact with portions of the inner layer 2 that include the release coating 4.

As shown in FIG. 3, once the absorbent pad 5 has been placed in the wrapper 1, the wrapper 1 is sealed along side seals 10 and 11. These side seals may be formed through any of the methods known in the art including ultrasonic bonding, heat sealing or adhesives. The wrapper 1 is maintained in a closed position through the use of a central closure mechanism 12. The closure mechanism 12 may use ultrasonic or thermal bonding, adhesives or mechanical fastening mechanisms.

When the absorbent pad 5 is ready to be used the closure mechanism 12 is opened and the absorbent pad 5 is removed from the wrapper 1. The release coating 4 reduces the amount of force needed to peel the wrapper 1 from the backsheet 8 and minimizes the amount of residual adhesive 9 that remains attached to the wrapper 1. The absorbent article 1 may then be attached to an article of clothing by the adhesive 9.

The release coating allows the nonwoven material of the wrapper of the present invention to release well from an adhesive strip. The release coating is a non-adherent treatment, generally comprising silicone or other low surface-energy substance, that facilitates peeling of the release paper or nonwoven overwrap of the invention from the pressure-sensitive adhesive on the backsheet of the absorbent article. The nonwoven material of the invention may be sprayed, immersed, wiped or otherwise contacted with the necessary amount of the release agent. The release coating may be applied continuously or intermittently. In the most preferred embodiment of the invention, the meltblown side of the spunbond/meltblown composite identified above, is sprayed or coated with a solution or suspension containing a high concentration of the release agent. The lower fluid permeability of the meltblown side of the nonwoven relative to the spunbond side thus minimizes the extent of release agent soaking through the composite.

In order to reduce the fluid permeability of the meltblown side of the preferred embodiment, the nonwoven material may undergo one or more treatments prior to application of the release coating. These treatments may include, but are not limited to, calendering, compression, heat treatment or application of a binder. U.S. Pat. No. 6,413,334 to Bodaghi provides an example of a nonwoven material treated to have a reduced fluid permeability.

In addition, the nonwoven material should remain hydrophobic, preferably before and after treatments, so that the wrapper can protect the absorbent pad from liquid. A suitable nonwoven for this application is comprised of polyolefins such as polypropylene or polyethylene, or other thermoplastics such as polyester or blends of these materials. In the preferred embodiment using a spunbond/meltblown laminate, the spunbond layer would provide the desired hydrophobic properties. Hydrophobic treatments may also be used to improve the hydrophobicity of the wrapper.

The nonwoven should not be too porous, and should have a minimum opacity and maximum permeability. It is important that when the nonwoven wrapper contacts the adhesive strip that the adhesive does not go through the nonwoven. This reduced porosity or permeability may also be helpful in efficiently coating the nonwoven with release agent. The nonwoven should have the appearance of being opaque, for several reasons. First, high opacity indicates that dirt and dust will not penetrate easily into the absorbent pad. High opacity will also provide superior capability to hide the absorbent product within the wrapper, which is important for discreetness. Also, high opacity enables the wrapper to appear as if it cannot be easily penetrated by outside fluid that could contaminate the absorbent pad, providing the user with more confidence.

The opacity of the nonwoven can be measured using ASTM E1347. The opacity, according to this test method, is preferably at least 20%, more preferably greater than 30%, and most preferably greater than 40%. Concurrently, the air permeability of the nonwoven, according to ASTM D737-96, is preferably no higher than 500 ft³/min, is more preferably less than 400 ft³/min and is most preferably less than 300 ft³/min. The hydrostatic head of the nonwoven, as measured according to INDA IST 80.6-98, is preferably at least 5 mbar, is more preferably at least 10 mbar, and is most preferably at least 15 mbar.

### Peel-Test Measurement

The nonwoven of the present invention can be detached from the adhesive strip with a low force, so as to be convenient and quiet. This force is referred to as the peel force. Preferably, the peel force of a product according to the invention does not exceed 300 g/in. More preferably, the force does not exceed 150 g/in, and it is most preferred to be less than 90 g/in.

The following test method is used to measure the peel force. The 90°-peel test measurement is executed on an INSTRON model 4443 equipped with a 90°-peel test fixture attached at the machine base instead of the lower grip. The support bar for the 90° pulley mounts to the top of the machine crosshead. In the test, the jaw is separated from the fixture at a constant rate of extension of 12″/min from an initial separation distance of 1″. The full-scale load is 5 N. The test samples are cut into 6″ long by 1″ wide strips. Then, 4″ lengths of the samples are marked to designate the areas to be tested. Next, 6″ long by 1.5″ wide strips of fastening tape KN1945 (3M Company, St. Paul, Minn.) are also cut. From one end of the fastening tapes, 1″ is folded over onto itself, creating an area where no adhesive is exposed. The 4″ marked lengths of the test samples are subsequently attached to the adhesive portions of the fastening tapes, with the remaining 2″ available to be gripped in the upper jaw of the INSTRON. Subsequently, a stainless steel cylinder weighing 9.8 lb and measuring 9 cm in diameter is rolled across each sample 3 times, making sure to eliminate air bubbles and wrinkles. Next, the samples are allowed to age underneath a 15″×15″×0.25″ plate of glass, loaded down in each corner of the plate with four of the aforementioned 9.8 lb stainless steel cylindrical weights for 24 hours.

After aging, the non-adhesive side of the fastening tape from the samples is secured to the test-fixture plate with double-sided adhesive tape. After positioning the test fixture directly under the grip and after loading the 2″ end of the test material into the grip, the peel test begins. The test samples are then peeled from the fastening tape, and the load at maximum load and the average load are recorded for the peel strength.

### Side-Seal Strength Measurement

The amount of force needed to detach the absorbent pad from the wrapper of the present invention should not exceed the amount of force needed to tear the side seals of the wrapper. The strength of the side seals is determined through a test method that uses the same apparatus and test fixture as the peel test described above. The INSTRON is set up the same way, too, except that the jaw separation is 2″. The samples are prepared by cutting a 1″ wide strip of material that is at least 2.5″ long from the sealed overwrap wrapper, making sure to include both layers of overwrap material in the cut. A 1 mm snip is then made at each end of the seal to ensure that at the end of the experiment, both layers of the overwrap are completely separated.

One side of the strip of side seal is affixed with double-stick tape to the test-fixture plate. The test fixture is then positioned directly underneath the upper grip. About 0.5″ of pre-peeled side seal is inserted into the upper grip, and then the peel separation begins. When the two layers of material are completely separated, the experiment ends. The load at maximum load and the average load are recorded for the side-seal strength.

### EXAMPLE 1

A nonwoven material, SM1702484, was obtained from First Quality Nonwovens, Inc., Great Neck, N.Y. The nonwoven is a 17 gsm fabric described as SMS, containing about 3.5 gsm of meltblown material. The material has an air permeability of about 250 ft³/min, an opacity of about 42% and a hydrostatic head of about 16.5 mbar. The nonwoven sample was immersed in a release agent, namely, C123/1 sold by Schill & Seilacher Group, Germany, diluted to a 50% concentration with water. Excess treatment was wrung out of the nonwovens, and the samples were left to dry overnight. After drying, ten samples were tested for peel strength as described above. The peel strengths measured were 75.6 g/in (load at maximum load) and 32.6 g/in average load.

In an additional experiment, the force required to open the side seals of a conventional nonwoven overwrap was measured. The nonwoven overwrap was taken from PREVAIL Ultra Bladder Control Pads, manufactured by First Quality Products, Inc. The seal strengths were measured as described above. Because the overwrap is folded, there are four sets of seals that can be tested from the wrapper: a first peel from either side and a second peel from either side. Ten seal strengths were measured for each of the four sets. The average of the forty seal strengths (load at maximum load) was 119.4 g/in. (load at maximum load) and 59.1 g/in (average load). Hence, the peel strengths measured for the treated nonwovens above are less than the side-seal strengths of the wrappers. This means that the amount of force required of a user to open the wrapper with nonwoven overwrap is less than or equal to the force required to peel off the overwrap from the pad. Hence, this comparative test establishes the requirement for easy removal of the releasable nonwoven wrappers made according to the invention.

I claim:

1. A wrapper for use with an absorbent article comprising:
a nonwoven material having a hydrostatic head of at least 5 mbar; and
a release agent applied to at least one side of said nonwoven material.

2. The wrapper of claim 1 wherein said nonwoven material comprises meltblown fibers.

3. The wrapper of claim 2 further comprising a layer of spunbond fibers.

4. The wrapper of claim 3 wherein said meltblown fibers and said spunbond fibers form a composite.

5. The wrapper of claim 1 wherein said release agent comprises a silicone-based agent.

6. The wrapper of claim 1 wherein said nonwoven material has a hydrostatic head of at least 10 mbar.

7. The wrapper of claim 1 wherein said nonwoven material has a hydrostatic head of at least 15 mbar.

8. A packaged absorbent article comprising:
an absorbent article comprising a topsheet, backsheet and absorbent core located between said topsheet and said backsheet;
an adhesive strip affixed to said backsheet; and
a wrapper for use with said absorbent article comprising
a nonwoven material having a hydrostatic head of at least 5 mbar; and
a release agent applied to at least one side of said nonwoven material,
wherein said adhesive strip is releasably attached to said treated nonwoven material.

9. The packaged absorbent article of claim 8 wherein said nonwoven material comprises meltblown fibers.

10. The packaged absorbent article of claim 9 wherein said wrapper further comprises spunbond fibers.

11. The packaged absorbent article of claim 10 wherein said meltblown fibers and spunbond fibers form a composite.

12. The packaged absorbent article of claim 8 wherein said release agent comprises a silicone-based agent.

13. The packaged absorbent article of claim 8 wherein the peel strength needed to detach said absorbent article from said wrapper is less than about 150 g/in.

14. The packaged absorbent article of claim 8 wherein the peel strength needed to detach said absorbent article from said wrapper is less than about 135 g/in.

15. The packaged absorbent article of claim 8 wherein the peel strength needed to detach said absorbent article from said wrapper is less than about 120 g/in.

16. The packaged absorbent article of claim 8 wherein the peel strength needed to detach said absorbent article from said wrapper is less than about 90 g/in.

17. The wrapper of claim 8 wherein said nonwoven material has a hydrostatic head of at least 10 mbar.

18. The wrapper of claim 8 wherein said nonwoven material has a hydrostatic head of at least 15 mbar.

19. A packaged absorbent article comprising:
an absorbent article comprising a topsheet, backsheet and absorbent core located between said topsheet and said backsheet;
an adhesive strip affixed to said backsheet; and
a wrapper for use with said absorbent article comprising side seals;
a nonwoven material having a hydrostatic head of at least 5 mbar, and;
a release agent applied to at least one side of said nonwoven material,
wherein said adhesive strip is releasably attached to said treated nonwoven material and the peel strength needed to detach said absorbent article from said wrapper is less than the peel strength needed to break said side seals.

20. The packaged absorbent article of claim 19 wherein said nonwoven material comprises meltblown fibers.

21. The packaged absorbent article of claim 20 wherein said wrapper further comprises spunbond fibers.

22. The packaged absorbent article of claim 21 wherein said meltblown fibers and said spunbond fibers form a composite.

23. The packaged absorbent article of claim 19 wherein said release agent comprises a silicone-based agent.

24. The packaged absorbent article of claim 19 wherein the peel strength needed to detach said absorbent article from said wrapper is less than about 150 g/in.

25. The packaged absorbent article of claim 19 wherein the peel strength needed to detach said absorbent article from said wrapper is less than about 135 g/in.

26. The packaged absorbent article of claim 19 wherein the peel strength needed to detach said absorbent article from said wrapper is less than about 120 g/in.

27. The packaged absorbent article of claim 19 wherein the peel strength needed to detach said absorbent article from said wrapper is less than about 90 g/in.

28. The wrapper of claim 19 wherein said nonwoven material has a hydrostatic head of at least 10 mbar.

29. The wrapper of claim 19 wherein said nonwoven material has a hydrostatic head of at least 15 mbar.
